SEQUENCE LISTING (12 sequences)

SEQ ID NO: 1 - DNA, 20 bp, synthetic construct
  source 1..20: mol_type = other DNA
gaggtgataaaattaactgc

SEQ ID NO: 2 - DNA, 19 bp, synthetic construct
  source 1..19: mol_type = other DNA
ggctctaccatatctccta

SEQ ID NO: 3 - DNA, 18 bp, synthetic construct
  source 1..18: mol_type = other DNA
catgtccagagctcattc

SEQ ID NO: 4 - DNA, 19 bp, synthetic construct
  source 1..19: mol_type = other DNA
gtttaaaatcactaggcga

SEQ ID NO: 5 - DNA, 19 bp, synthetic construct
  source 1..19: mol_type = other DNA
ctgaccatttccatcattc

SEQ ID NO: 6 - DNA, 18 bp, synthetic construct
  source 1..18: mol_type = other DNA
gtaactaaacaggagccg

SEQ ID NO: 7 - DNA, 21 bp, synthetic construct
  source 1..21: mol_type = other DNA
atgtatccattgagcattgcc

SEQ ID NO: 8 - DNA, 20 bp, synthetic construct
  source 1..20: mol_type = other DNA
cacgaatcagcggtaaaggt

SEQ ID NO: 9 - DNA, 21 bp, synthetic construct
  source 1..21: mol_type = other DNA
gagatatggtagagccgcaga

SEQ ID NO: 10 - DNA, 24 bp, synthetic construct
  source 1..24: mol_type = other DNA
tttcagcagctacagtcagaattt

SEQ ID NO: 11 - DNA, 25 bp, synthetic construct
  source 1..25: mol_type = other DNA
cgatgggttaattcgctcgttgtgg

SEQ ID NO: 12 - DNA, 20 bp, synthetic construct
  source 1..20: mol_type = other DNA
gcacaacggaaagagcactg

(19) United States
(12) Patent Application Publication
(10) Pub. No.: US 2023/0392190 A1
(43) Pub. Date: Dec. 7, 2023

(54) METHOD AND KIT FOR DETECTING CELL-FREE DNA METHYLATION

(71) Applicant: DALIAN GENTALKER BIO-TECH CO., LTD., Dalian (CN)

(72) Inventors: Hongzhi LI, Dalian (CN); Qi LIU, Dalian (CN); Jinyin ZHAO, Dalian (CN); Xinyu DUAN, Dalian (CN); Lizhi XU, Dalian (CN); Jie LI, Dalian (CN)

(21) Appl. No.: 18/337,131

(22) Filed: Jun. 19, 2023

Related U.S. Application Data
(63) Continuation of application No. PCT/CN2022/111606, filed on Aug. 11, 2022.

(30) Foreign Application Priority Data
Dec. 1, 2021 (CN) 202111455145.7

(51) Int. Cl. C12Q 1/6806; C12Q 1/6869; C12Q 1/6886

(52) U.S. Cl. CPC C12Q 1/6806; C12Q 1/6869; C12Q 1/6886; C12Q 2600/154

(57) ABSTRACT

The present disclosure provides a method and a kit for detecting cell-free DNA (cfDNA) methylation, and belongs to the technical field of early cancer screening. The method includes the following steps: constructing and mixing a cfDNA library and a filter DNA to obtain a mixture of the cfDNA library and the filter DNA, co-immunoprecipitating the mixture with anti-5-methylcytosine (5mC) antibody, conducting methylation capture on methylated DNA fragments in the mixture, and purifying and eluting to obtain a captured product fragment; conducting amplification and enrichment, purification, recovery and screening to obtain a sequencing library; and sequencing on an Illumina sequencing platform, and bioinformatically analyzing acquired experimental data to know about the cfDNA methylation. The detection method and the kit provided by the present disclosure feature high detection sensitivity and low experimental cost, and substantially reduce the false positive rate of conventional detection to obtain more reliable results.

### CROSS REFERENCE TO RELATED APPLICATION

This patent application is a national stage application of International Patent Application No. PCT/CN2022/111606, filed on Aug. 11, 2022, which claims the benefit and priority of Chinese Patent Application No. CN202111455145.7 filed with the China National Intellectual Property Administration on Dec. 1, 2021, and entitled “METHOD AND KIT FOR DETECTING CELL-FREE DNA (cfDNA) METHYLATION”, the disclosure of which is incorporated by reference herein in its entirety as part of the present application.

A sequence listing is submitted in XML format via the USPTO patent electronic filing system and is herein incorporated by reference in its entirety. The name of the sequence listing file is “Sequence Listing.xml” created on Aug. 10, 2022, and has a file size of 11,000 bytes (11 kb).

### TECHNICAL FIELD

The present disclosure belongs to the technical field of early cancer screening, and in particular relates to a novel method and a kit for detecting cell-free DNA (cfDNA) methylation.

### BACKGROUND

Malignant tumors, commonly known as cancers, are diseases caused by the loss of normal regulation and excessive proliferation of somatic cells. Cancer cells can develop in most organs and tissues in the body, invade surrounding tissues, and even metastasize to other parts of the body through the in vivo circulatory/lymphatic system. According to statistics, cancer is the second leading cause of death globally, with approximately 18 million new cases and 9.6 million deaths in 2018. By 2030, it is expected that there will be 26 million new cases and 17 million deaths throughout the year, posing a serious threat to human life and health. Advanced cancers usually lack effective therapies, but if cancers are detected at an early stage, the survival rate will be significantly improved, with a five-year survival of about 91%. Detecting tumors at the earliest possible stage is the key to treatment. In recent years, cfDNA has emerged as a promising tumor biomarker in early cancer diagnosis research with great potential for early diagnosis.

Research on the mechanisms of the pathogenesis, progression, and metastasis of cancers is based on different platforms, involving genomes, transcriptomes, proteomes, metabolomes, and epigenomes. Recently, the role of the epigenomes in normal and cancer cells has been demonstrated and made rapid progress, and the epigenomes mainly regulated by DNA methylation and chromatin configuration regulate gene expression by altering nucleosome structure and mapping. In normal human cells, nucleosomes maintain an open conformation without DNA methylation sites in the promoter region, whereas in tumors the nucleosome spacing is relatively closed. It has been shown that DNA methylation has been defined as key events in the pathogenesis and progression of cancers. DNA methylation occurs at CpG locus, at which a methyl group is added to the 5′-C position of the cytosine base to form 5-methylcytosine (5mC) in the presence of DNA methyltransferases (DNMTs). DNA methylation patterns are frequent in cancers, including DNA demethylation events at retroelements, centromeres, and oncogenes. Change in 5mC has the ability to distinguish cancer cells from normal cells, and epigenetic profile thereof can be used as a plurality of tumor markers for early diagnosis and prognosis monitoring, and has become a research hotspot in gene detection.

Conventional DNA methylation detection generally adopts bisulfite treatment followed by next-generation sequencing (NGS). This method has limited detection sensitivity, low reliability of results, and high detection costs due to the limited amount of cfDNA, the loss of degradation of about 84% of DNA caused by bisulfite, a low genome-wide abundance of CpGs, and the limited information recovery rate.

### SUMMARY

To solve the foregoing technical problems, an objective of the present disclosure is to provide a method and a kit for detecting cfDNA methylation based on immunoprecipitation. The detection method provided by the present disclosure is sensitive and reliable, and can be used for early cancer screening without bisulfite treatment.

The objective of the present disclosure is achieved by the following technical solutions:

The present disclosure provides a construction method of a sequencing library for cfDNA methylation, mainly including the following steps:
step 1, extracting whole blood cfDNA, and constructing a cfDNA library by end repair, A-tailing, and ligation of Illumina sequencing platform-specific index adapters;
step 2, mixing the cfDNA library constructed in step 1 with a filler DNA constructed in advance to obtain a mixture of the cfDNA library and the filler DNA, in order to ensure that an initial input reaches at least 100 ng;
step 3, co-immunoprecipitating anti-5-methylcytosine (5mC) antibody with the mixture of the cfDNA library and the filler DNA obtained in step 2, conducting methylation capture on differentially methylated DNA fragments in the mixture, and purifying and eluting to obtain captured product fragments;
step 4, conducting amplification and enrichment on the product fragments obtained in step 3, and purifying, recovering and screening amplified products with AMPure XP Beads to obtain a final sequencing library; and
step 5, sequencing the final sequencing library on an Illumina sequencing platform, and bioinformatically analyzing acquired experimental data to know about the cfDNA methylation.

Further, the cfDNA in step 1 is extracted and obtained by a QIAamp Circulating Nucleic Acid Kit.

Further, the end repair and the A-tailing in step 1 are completed by means of an End Repair & A-Tailing Enzyme Mix reaction system.

Further, no sequencing adapter is added to the filler DNA in step 2, only aiming to expand the initial input of cfDNA sequencing.

Further, the filler DNA in step 2 is composed of six polymerase chain reaction (PCR) amplicons of different sizes and CpG densities (1 CpG, 5 CpG, 1 CpG, 15 CpG, 20 LCpG, and 20 SCpG), five fragments of different CpG densities (1 CpG, 5 CpG, 1 CpG, 15 CpG, and 20 LCpG fragments) are methylated, and one fragment (20 SCpG fragment) is unmethylated.

Further, the filler DNA in step 2 is obtained by performing a PCR with XDNA as a template, purifying and recovering, and methylating, purifying and recovering a resulting PCR fragment.

Further, the filler DNA in step 2 is composed of 50% (wt/wt) methylated fragments (1 CpG, 5 CpG, 1 CpG, 15 CpG, and 20 CpGL fragments) and a 50% (wt/wt) unmethylated fragment (20 CpGS PCR amplification).

Further, the methylation capture in step 3 is completed by means of a Diagenode MagMeDIP Kit and a Diagenode iPure Kit V2.

Further, the amplification and enrichment in step 4 is performed by ligation-mediated polymerase chain reaction (LM-PCR).

Further, the Illumina sequencing platform in step 5 is one selected from the group consisting of Illumina NextSeq 500, Illumina Hiseq2000, Illumina Hiseq2500, and Illumina Miseq.

An aspect of the present disclosure provides a method for detecting cfDNA methylation, including steps of: sequencing a sequencing library obtained by the construction method on an Illumina sequencing platform, and bioinformatically analyzing acquired experimental data to know about the cfDNA methylation.

An aspect of the present disclosure provides a kit for the foregoing method for detecting cfDNA methylation. The kit includes the following components: components for NGS library preparation, a filler DNA fragment, components for co-immunoprecipitation, methylation capture, and purification and recovery, and components for library enrichment.

Further, the components for NGS library preparation mainly include commonly used enzymes desired for end repair, A-tailing and adapter ligation in the NGS library preparation and Illumina sequencing platform-specific adapters.

Further, the filler DNA fragment is composed of six PCR amplicons of different sizes and CpG densities (1 CpG, 5 CpG, 1 CpG, 15 CpG, 20 LCpG, and 20 SCpG), five fragments of different CpG densities (1 CpG, 5 CpG, 1 CpG, 15 CpG, and 20 LCpG fragments) are methylated, and one fragment (20 SCpG fragment) is unmethylated.

Further, the filler DNA fragment is obtained by performing a PCR with XDNA as a template, purifying and recovering, and methylating, purifying and recovering a resulting PCR fragment.

Further, primers desired for the PCR have nucleotide sequences shown in SEQ ID NOs: 1 to 12.

Further, the components for co-immunoprecipitation mainly include buffer reagents desired for the co-immunoprecipitation, an antibody protein, magnetic beads for the methylation capture, and reagents and an Elution Buffer desired for the purification and recovery.

Further, the components for library enrichment mainly include an enzyme and a buffer desired for library amplification, and magnetic beads desired for product recovery and purification and fragment screening.

The present disclosure further provides use of the foregoing kit in early pan-cancer screening.

The present disclosure further provides use of the foregoing kit in early lung cancer screening.

Further, a risk of the early lung cancer screening is determined based on analytical data of differentially methylated regions (DMRs) and methylation levels of samples.

The present disclosure has the following beneficial effects:

The method for detecting cfDNA methylation provided by the present disclosure avoids the degradation loss of DNA caused by bisulfite treatment. Different from conventional methylation sequencing methods, the present disclosure is independent of bisulfite. The core is methylated DNA immunoprecipitation, by which methylated DNA fragments are specifically captured by using methylated anti-5-mC antibody, so that all methylated DNAs in a sample are precipitated and enriched. All samples obtained are fractions with methylated DNA in a screened genome, so that reaction specificity can reach 99%, detection sensitivity is high, and experimental costs are reduced. Meanwhile, the false positive rate of conventional detection is substantially reduced to obtain more reliable results.

### BRIEF DESCRIPTION OF THE DRAWINGS

FIG. 1 is an experimental flow chart of a method for detecting cfDNA methylation provided by the present disclosure;

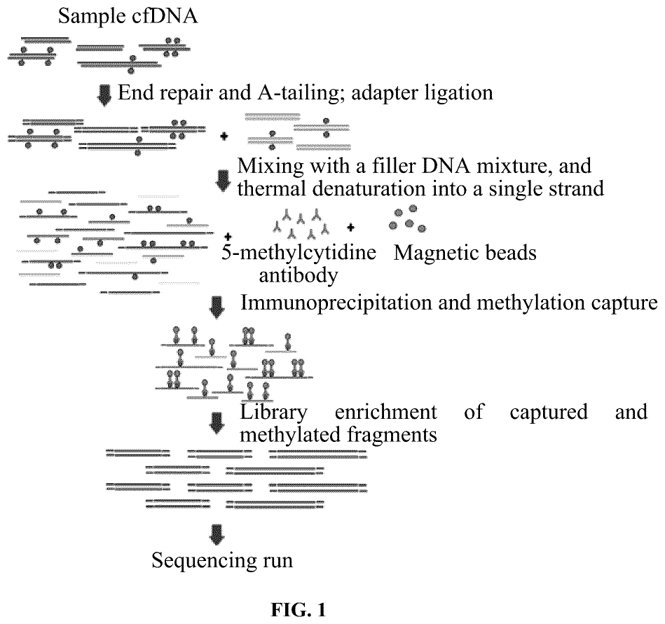

FIG. 2 is a statistical chart of a sequencing depth of DMRs of sample S1-1; and

FIG. 3 is a statistical chart of a sequencing depth of DMRs of sample S1-2.

### DETAILED DESCRIPTION OF THE EMBODIMENTS

The implementations of the present disclosure will be described in detail below with reference to examples, but those skilled in the art will understand. In various implementations of the present disclosure, a plurality of technical details are set forth in order to provide the reader with a better understanding of the present application. However, even without these technical details, the technical solutions protected by the claims of the present application can also be implemented.

Equipment and reagents used in the following examples are as follows: QIAamp Circulating Nucleic Acid Kit (QIAGEN, Germany), ABI 2720 Thermal Cycler, MeDIP Kit (Diagenode, Belgium), library preparation kit (Kapa Biosystems, USA), and sequencing platform Illumina NextSeq 500.

### Example 1: Preparation of a Filler DNA Mixture

The filler DNA could be prepared in batches according to the experimental scale in advance and stored at −20° C. The filler DNA with sequencing adapter was not included in the library. The purpose was to expand the initial input in cfDNA sequencing, so it had no effect on the subsequent sequencing results. The specific operation procedure was as follows.

1. Design of Primers Desired for Filler DNA Preparation

In order to solve the problems of limited cfDNA quantity and low mutation rate of cfDNA methylation, the present disclosure substantially increased the initial input in cfDNA sequencing by preparing a filler DNA/library mixture, so that the input reached 100 ng. The filler DNA was composed of six PCR amplicons of different sizes and CpG densities (1 CpG, 5 CpG, 10 CpG, 15 CpG, 20 LCpG, and 20 SCpG). They were composed of fragments of Enterobacteria phage (λ-DNA), which were produced by PCR and methylated (at appropriate loci) in vitro. Five fragments with different CpG densities were methylated, and one fragment was unmethylated. They had no common homology with mammalian genomes. The primers desired for filler DNA preparation are shown in Table 1.

TABLE 1
List of primers desired for filler DNA preparation

| Name | Forward Primer (5′ to 3′) | Reverse Primer (5′ to 3′) |
|---|---|---|
| 1CpG | GAGGTGATAAAATTAACTGC | GGCTCTACCATATCTCCTA |
| 5CpG | CATGTCCAGAGCTCATTC | GTTTAAAATCACTAGGCGA |
| 10CpG | CTGACCATTTCCATCATTC | GTAACTAAACAGGAGCCG |
| 15CpG | ATGTATCCATTGAGCATTGCC | CACGAATCAGCGGTAAAGGT |
| 20CpGL | GAGATATGGTAGAGCCGCAGA | TTTCAGCAGCTACAGTCAGAATTT |
| 20CpGS | CGATGGGTTAATTCGCTCGTTGTGG | GCACAACGGAAAGAGCACTG |

2. Filler DNA Preparation

The filler DNA fragment was prepared by PCR with XDNA as a template. The reaction system is shown in Table 2:

TABLE 2
T reaction system for filler DNA preparation

| Component | Volume desired for reaction | |
|---|---|---|
| Platinum SuperFi PCR Master Mix (2×) | 25 | μL |
| Forward primer (10 μM) (Table 1) | 1.5 | μL |
| Reverse primer (10 μM) (Table 1) | 1.5 | μL |
| Nuclease-free water | 21 | μL |
| Diluted λDNA (0.1 ng/μL) | 1 | μL |
| Total | 50 | μL |

Forward and reverse primers are primers for 1 CpG, 5 CpG, 10 CpG, 15 CpG, 20 CpGL, and 20 CpGS provided in Table 1. The above six primer pairs were amplified by PCR separately. After the reaction system was prepared, it was mixed well, centrifuged, and placed in a PCR system, on which the PCR was carried out under the following reaction conditions: holding at a hot lid temperature of 105° C., initial denaturation at 98° C. for 30 s; 30 cycles of denaturation at 98° C. for 10 s, annealing at 57° C. for 10 s, and extension at 72° C. for 15 s; and extension at 72° C. for 5 min and holding at 4° C.

After the PCR, the PCR products were purified and recovered by the Tiangen Universal DNA Purification Kit, and eluted with 30 μL of ultrapure water. The fragment size was verified by Qseq, followed by Qubit quantification to obtain selected PCR fragments.

3. Methylation of the Selected PCR Fragments

PCR fragments of 1 CpG, 5 CpG, 1 CpG, 15 CpG, and 20 CpGL amplified in the previous step were each methylated. The reaction system is shown in Table 3:

TABLE 3
Reaction system for methylation of PCR fragments

| Component | Volume desired for reaction | |
|---|---|---|
| PCR amplicon (up to 1 μg) | Up to 16.6 μL | |
| 10× M.SssI buffer | 2 | μL |
| 50× SAM (provided with CpG M.SssI) | 0.4 | μL |
| M.SssI enzyme | 1 | μL |
| Total | 20 | μL |

The reaction system was placed in the PCR system to carry out the following program: 37° C. for 15 min, and 65° C. for 20 min.

After the reaction, the product was purified and recovered by the Tiangen Universal DNA Purification Kit, eluted with 30 μL of pure water, and quantified by Qubit.

The filler DNA was ultimately composed of 50% (wt/wt) methylated fragments (1 CpG, 5 CpG, 10 CpG, 15 CpG, and 20 CpGL fragments) and a 50% (wt/wt) unmethylated fragment (20 CpGS PCR amplification). The products of the fragments were mixed in the ratio shown in Table 4 to obtain a filler DNA mixture.

TABLE 4
Composition of filler DNA mixture

| Methylation pattern | Fragment name | Total quantity desired | |
|---|---|---|---|
| Methylated | 1CpG | 10 | ng |
| | 5CpG | 10 | ng |
| | 10CpG | 10 | ng |
| | 15CpG | 10 | ng |
| | 20CpGL | 10 | ng |
| Unmethylated | 20CpGS | 50 | ng |
| | Total | 100 | ng |

### Example 2: Detection of Methylation of Plasma cfDNA Samples from Two Patients with Lung Cancer

In cooperation with a hospital, plasma samples were collected from two cancer patients, and the methylation of cfDNA of the plasma samples of the patients was detected by the method provided in this application to illustrate the feasibility and practicability of this patent. The specific operation procedure was as follows:

1. Sample Collection, Delivery, and Storage

The samples of the present disclosure were selected from human whole blood. 3 mL of venous blood was collected from each of the two cancer patients (numbered as S1-1 and S1-2) in a collection tube filled with ethylenediaminetetraacetic acid (EDTA)/anticoagulant acid citrate dextrose (ACD-A). Samples were transported to the laboratory as soon as possible at room temperature. The samples should be stored at 2-8° C. for no more than three days and at −20° C. for no more than a month, and those that needed to be stored for a long time should be stored at −80° C. Genomic DNA extraction should be completed as soon as possible as of the date of sample collection.

2. Extraction of Plasma cfDNA

Plasma cfDNA samples were extracted from two patients numbered S1-1 and S1-2, respectively. The concentration of each extracted sample was quantified by Qubit3.0, and the extracted samples were temporarily stored in a −20° C. freezer.

The extraction method of plasma cfDNA was implemented with reference to the instructions of the QIAamp Circulating Nucleic Acid Kit (Qiagen). The operation procedure was as follows:
1) 1 mL of plasma sample was pipetted to a clean 50 mL centrifuge tube with a pipette, marked well, supplemented with 200 μL of Proteinase K and 0.8 mL of ATP-citrate lyase (ACL), vortexed for 30 s, and incubated in a 60° C. water bath for 30 min;
2) 1.8 mL of ACB Buffer (confirmed that isopropanol had been added) was added, and the centrifuge tube was vortexed for 30 s and incubated on ice for 5 min;
3) a small 20 mL expander was inserted into mini columns, and the mini columns were inserted into a vacuum generator for use; the solution obtained in step 2) was poured into the expander, the vacuum pump was turned on (at a vacuum pressure ranging from −200 to −800 MPa), the liquid was suctioned dry, and the vacuum pump was turned off, followed by marking;
4) 600 μL of ACW1 (confirmed that absolute ethanol was added) was added into the expander, the vacuum pump was turned on (at a vacuum pressure ranging from −200 to −800 MPa), the liquid was suctioned dry, and the vacuum pump was turned off,
5) 750 μL of ACW2 (confirmed that absolute ethanol was added) was added into the expander, the vacuum pump was turned on (at a vacuum pressure ranging from −200 to −800 MPa), the liquid was suctioned dry, and the vacuum pump was turned off,
6) 750 μL of absolute ethanol (96%-100%) was added into the expander, the vacuum pump was turned on (at a vacuum pressure ranging from −200 to −800 MPa), the liquid was suctioned dry, and the vacuum pump was turned off,
7) the expander was discarded, and the mini columns were left in a 2 mL centrifuge tube and centrifuged at 14,000 rpm for 3 min;
8) the centrifuge tube was put in a 56° C. metal bath to dry for 10 min (unlidded);
9) the mini columns were put into a new 1.5 mL centrifuge tube, and 55 μL of water was added and let stand at room temperature for 5 min; note: the elution buffer AVE was equilibrated to room temperature (15-25° C.) and must be distributed to the center of the membrane; and
10) the cfDNA was eluted by centrifugation at 14,000 rpm for 1 min.

3. Library Preparation

Plasma cell-free DNA samples were subjected to library preparation experiments. After end repair, A-tailing and ligation, the two groups of DNA fragments were ligated to different index adapters (where relevant reagents were from KAPA Hyper Prep Kit Illumina platforms). The specific procedure was slightly modified on the basis of the library preparation kit protocol. The steps were as follows:

1) End Repair and 3′-End A-Tailing, where the Reaction System is as Shown in Table 5:

TABLE 5
Reaction system for end repair and 3′-end A-tailing

| Component | Volume | |
|---|---|---|
| cf DNA | 35 | μL |
| Nuclease-free water | 15 | μL |
| End Repair & A-Tailing buffer | 7 | μL |
| End Repair & A-Tailing Enzyme Mix | 3 | μL |
| Total | 60 | μL |

The reaction system was pipetted to mix well (to avoid vigorous shaking), and centrifuged briefly; and
the reaction program was: holding at a hot lid temperature of 85° C., 20° C. for 30 min; 65° C. for 30 min; and holding at 4° C.

2) Adapter Ligation

In the PCR tube for the above reaction, the reaction system was prepared on an ice box as shown in Table 6:

TABLE 6
Reaction system for adapter ligation

| Component | Volume | |
|---|---|---|
| Reaction product for end repair and A-tailing | 60 | μL |
| Nuclease-free water | 2.5 | μL |
| Methyl Adapter | 7.5 | μL |
| Ligation Buffer | 30 | μL |
| DNA Ligase | 10 | μL |
| Total | 110 | μL |

The reaction system was pipetted to mix well (to avoid vigorous shaking), and centrifuged briefly; and
the reaction program was: closing the hot lid; 20° C. for 15 min; and holding at 4° C.

3) Purification after Ligation:
i) After the PCR, 88 μL of Agencourt AMPure XP Beads were added to the sample and pipetted to mix well;
ii) after incubation at room temperature for 5 min, the PCR tube was placed on a magnetic rack for 3 min until the solution became clear;
iii) the PCR tube was held on the magnetic rack, the supernatant was discarded, and 200 L of 80% ethanol solution was added to the PCR tube and let stand for 30 s;
iv) the PCR tube was held on the magnetic rack, the supernatant was discarded, 200 μL of 80% ethanol solution was added to the PCR tube and let stand for 30 s, and the supernatant was completely removed;
v) the PCR tube was incubated at room temperature for 5 min to completely volatilize the residual ethanol;
vi) 42 μL of Nuclease-free Water was added, and the PCR tube was removed from the magnetic rack and pipetted to mix well;
vii) after incubation at room temperature for 2 min, the PCR tube was let stand on the magnetic rack for 2 min until the solution became clear; and
viii) 40 μL of supernatant was pipetted and transferred to a new PCR tube, and the sample information was labeled to obtain a ligated library product.

4. Methylation Capture

The reagents desired for methylation capture experiment were from Diagenode MagMeDIP Kit and Diagenode iPure Kit V2. The specific steps were as follows:

(1) Reagent Preparation
1) 5×Mag Buffer was diluted to the concentration of working solution according to the following table:

TABLE 7
Dilution ratio of 5× Mag Buffer

| Component | Volume desired for reaction | |
|---|---|---|
| 5× Mag Buffer | 20 | μL |
| Nuclease-free water | 80 | μL |
| Total | 100 | μL |

2) 11 μL of magnetic beads were pipetted into a new Eppendorf (EP) tube, the EP tube was placed on a magnetic rack until clear, and the supernatant was discarded.
3) Magnetic beads were cleaned twice with 27.5 μL of 1×Mag Buffer on an ice bath. Then, magnetic beads were resuspended with 22 μL of 1×MagBuffer, transferred to a new EP tube and placed on ice for later use.
4) The reagent Mag master mix was prepared according to the table below:

TABLE 8
Preparation ratio of Mag master mix

| Component | Volume desired for reaction | |
|---|---|---|
| 5× Mag Buffer A | 24 | μL |
| Mag Buffer B | 6 | μL |
| Nuclease-free water | 3 | μL |
| Total | 33 | μL |

5) The anti-5-mC antibody reagent was half-diluted to prepare an antibody reaction buffer in the following ratio:

TABLE 9
Preparation ratio of antibody reaction buffer

| Component | Volume desired for reaction | |
|---|---|---|
| Half-diluted anti-5-mC antibody | 0.3 | μL |
| 5× Mag Buffer A | 0.6 | μL |
| Mag Buffer C | 2.1 | μL |
| Nuclease-free water | 2 | μL |
| Total | 5 | μL |

(2) Immunoprecipitation
1) The mixture of cfDNA library and filler DNA prepared in a ratio was mixed with the Mag master mix prepared in the previous step in a 0.2 mL PCR tube in the following ratio, and shaken to mix well.

TABLE 10
Mixing ratio of cfDNA library to filler DNA

| Component | Volume desired for reaction | |
|---|---|---|
| cfDNA library/filler DNA mixture (100 ng) | x | μL |
| Mag master mix | 33 | μL |
| Nuclease-free water | 57 − x | μL |
| Total | 90 | μL |

The well-mixed PCR tube was placed on a PCR system, denatured at 95° C. for 3 min, and placed on ice immediately, and 75 μL of the mixture was transferred into a new PCR tube.
2) 5 μL of the prepared antibody reaction buffer was added to the PCR tube.
3) 20 μL of prepared magnetic beads components were added to the PCR tube, mixed well, and incubated overnight at 4° C. under shaking.

(3) Purification and Recovery of Methylated DNA Fragments
1) Elution Buffer was prepared according to the following table (Buffer A needed to be let stand at room temperature for 30 min before use):

TABLE 11
Preparation of Elution Buffer

| Component | Volume desired for reaction | |
|---|---|---|
| Buffer A | 115.4 | μL |
| Buffer B | 4.6 | μL |
| Total | 120 | μL |

2) 50 μL of Elution Buffer was added to the PCR tube for the immunoprecipitation in the previous step, mixed well, and incubated at room temperature for 15 min.
3) The PCR tube was placed on a magnetic rack for 1 min, and the supernatant was transferred to a new EP tube.
4) 50 μL of Elution Buffer was added to the original PCR tube to mix the magnetic beads well, and incubated at room temperature for 15 min. The PCR tube was placed on the magnetic rack for 1 min, and the supernatant was transferred to an EP tube.
5) 2 μL of Carrier was added to the EP tube, vortexed briefly and centrifuged transiently.
6) 100 μL of isopropanol was added to the EP tube, vortexed briefly and centrifuged transiently.
7) 10 μL of magnetic beads were added to the EP tube, mixed well and incubated at room temperature under shaking for 10 min.
8) The EP tube was placed on the magnetic rack for 1 min, the supernatant was pipetted and discarded, 25 μL of Buffer C was added to the tube, inverted and mixed to resuspend the magnetic beads, and the EP tube was incubated at room temperature under shaking for 15 min.
9) The EP tube was placed on the magnetic rack for 1 min, and 25 μL of the supernatant was pipetted into a new EP tube to obtain methylated and captured DNA fragments for downstream experiments.

5. Library Amplification, Enrichment, Purification and Screening after Methylation Capture

The cfDNA library fragments obtained after methylation capture in the previous step were subjected to the LM-PCR enrichment operation of the samples. The LM-PCR system is shown in the following table:

TABLE 12
LM-PCR system

| Component | Volume | |
|---|---|---|
| KAPA HiFi HotStart ReadyMix | 25 | μL |
| Post-LM-PCR Oligos 1 & 2.5 μM | 5 | μL |
| Magnetic bead solution with captured fragments | 20 | μL |
| Total | 50 | μL |

The PCR program was: initial denaturation at 98° C. for 45 s; 14 cycles of denaturation at 98° C. for 15 s, annealing at 60° C. for 30 s, and extension at 72° C. for 30 s; extension at 72° C. for 1 min; and holding at 4° C.

After the PCR, the PCR products were purified by using AMPure Beads, and fragments were screened to obtain a sample library, which was used for sequencing analysis after quality inspection. Specific steps were as follows:
i) Agencourt AMPure XP Beads were taken out of the 4° C. refrigerator and equilibrated to room temperature, vortexed, and shaken to mix well before use; 50 μL of magnetic beads were added to the PCR products in the previous step, pipetted to mix well, and let stand at room temperature for 5 min; and the PCR tube was placed on a magnetic rack for 3 min until the solution became clear;
ii) the PCR tube was held on the magnetic rack, the supernatant was discarded, and 200 L of 80% ethanol solution was added to the PCR tube and let stand for 30 s;
iii) the PCR tube was held on the magnetic rack, the supernatant was discarded, additional 200 μL of 80% ethanol solution was added to the PCR tube and let stand for 30 s, and the supernatant was completely removed;
iv) the PCR tube was let stand at room temperature for 3-5 min to completely volatilize the residual ethanol;
v) 32 μL of Nuclease-free Water was added, and the PCR tube was removed from the magnetic rack, pipetted to mix well, and let stand for 2 min;
vi) the PCR tube was let stand on the magnetic rack for 2 min until the solution became clear, and 30 μL of the supernatant was pipetted, transferred to a new PCR tube, and labeled;
vii) 15 μL of magnetic beads were added to the PCR tube, pipetted to mix well, and let stand at room temperature for 5 min; and the PCR tube was let stand on the magnetic rack for 3 min until the solution became clear;
viii) the PCR tube was held on the magnetic rack, the supernatant was pipetted into a new PCR tube, 12 μL of magnetic beads were added to the tube, pipetted to mix well, and let stand at room temperature for 5 min; and the PCR tube was let stand on the magnetic rack for 3 min until the solution became clear;
ix) the PCR tube was held on the magnetic rack, the supernatant was discarded, and 200 L of 80% ethanol solution was added to the PCR tube and let stand for 30 s;
x) the PCR tube was held on the magnetic rack, the supernatant was discarded, additional 200 μL of 80% ethanol solution was added to the PCR tube and let stand for 30 s, and the supernatant was completely removed;
the PCR tube was let stand at room temperature for 3-5 min to completely volatilize the residual ethanol;
32 μL of Nuclease-free Water was added, and the PCR tube was removed from the magnetic rack, pipetted to mix well, and let stand for 2 min;
the PCR tube was let stand on the magnetic rack for 2 min until the solution became clear, and 30 μL of the supernatant was pipetted, transferred to a new PCR tube, and labeled; and
after quantification by Qubit and fragment quality inspection by Qseq, subsequent sequencing experiment was performed, or the library was stored in a −20° C. freezer.

6. NGS and Result Analysis

A sample sequencing library after methylation capture was prepared by the above method. Sequencing was conducted using the pair-End sequencing technology of the Illumina sequencing platform, such as Illmina NextSeq 500, Illumina Hiseq2000, Illumina Hiseq2500 and Illumina Miseq, to obtain sequences of the DNA mixture. Each sample required at least 30 M reads.

The analysis process started with the basic QC that analyzes raw reads in FastQC, followed by trimming of adapter contamination using Trim Galore. The trimmed data were aligned to the reference genome using BWA-mem or Bowtie 2, and the acquired SAM files were converted to BAM file format using SAMtools. Afterwards, using bioinformatics analysis, the sequencing depth of 14,716 DMRs on the human genome was statistically analyzed to generate DMR analysis data and scores of sample methylation levels. The methylation level of the sample was evaluated according to the established data model. The results of two cfDNA samples were analyzed as follows:

TABLE 13
Analytical results after sample sequencing

| | | Number of | | | Average | |
|---|---|---|---|---|---|---|
| Sample | Acquired | acquired | | Coverage | depth of | Scoring |
| No. | data size | reads | Q30 | of DMR | DMRs | result |
| S1-1 | 6.53 G | 43,553,333 | 79.33 | 85.06% | 8.26 | High risk |
| S1-2 | 6.96 G | 46,386,666 | 80.37 | 83.2% | 9.68 | High risk |

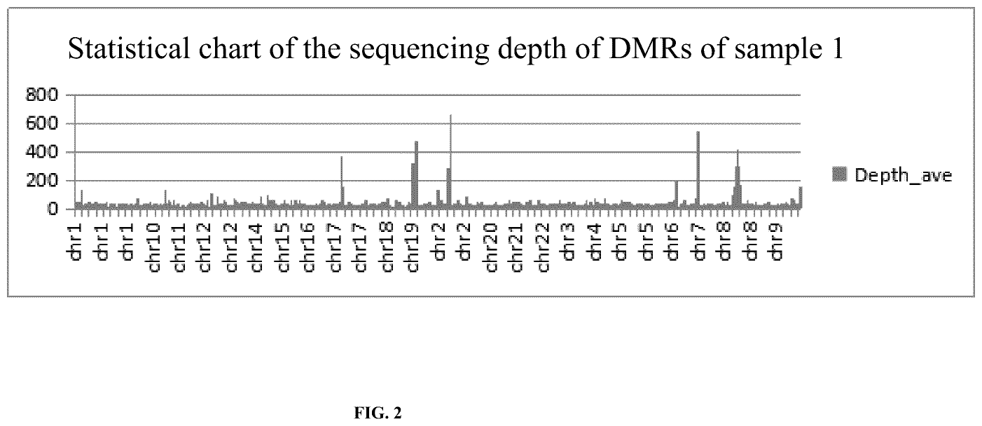

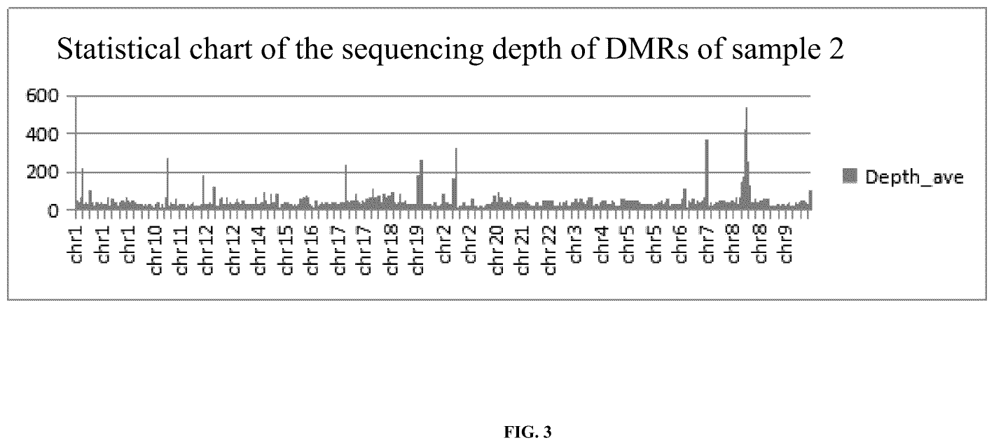

The method for detecting methylation provided by the present disclosure can better preserve the methylation status of the sample. Therefore, the detection result is more accurate and reliable, the degradation loss of the sample DNA can be reduced during the detection, and the detection sensitivity and specificity can be substantially improved. According to the analytical results in Table 13, the detection results of the two clinical samples are reliable, the quality of the sequencing data is good, and the detection method provided by the present disclosure is accurate and effective. FIGS. 2 and 3 show the depth of 14,716 methylated regions in two samples. According to the bioinformatics analysis method and the big data model of early cancer screening, the two clinical samples are evaluated as high-risk results, which are consistent with clinical information.

Finally, it should be noted that the foregoing examples are only intended to illustrate the technical solutions of the present disclosure, but not to limit them; although the present disclosure has been described in detail with reference to the foregoing examples, those of ordinary skill in the art should understand that the technical solutions described in the foregoing examples can still be modified, or some or all of the technical features thereof can be equivalently substituted; and these modifications or substitutions do not make the essence of the corresponding technical solutions depart from the scope of the technical solutions of the examples of the present disclosure.

What is claimed is:

1. A construction method of a sequencing library for cell-free DNA (cfDNA) methylation, comprising the following steps:
step 1, extracting whole blood cfDNA, and constructing a cfDNA library by end repair, A-tailing, and ligation of Illumina sequencing platform-specific index adapters;
step 2, mixing the cfDNA library constructed in step 1 with a filler DNA constructed in advance to obtain a mixture of the cfDNA library and the filler DNA;
step 3, co-immunoprecipitating anti-5-methylcytosine (5mC) antibody with the mixture of the cfDNA library and the filler DNA obtained in step 2, and capturing and purifying methylated DNA fragments in the mixture to obtain captured product fragments; and
step 4, conducting amplification and enrichment on the product fragments obtained in step 3, and purifying, recovering and screening amplified products with magnetic beads to obtain a final sequencing library.

2. The construction method according to claim 1, wherein the cfDNA in step 1 is extracted and obtained by a QIAamp Circulating Nucleic Acid Kit.

3. The construction method according to claim 1, wherein the end repair and the A-tailing in step 1 are completed by means of an End Repair & A-Tailing Enzyme Mix reaction system.

4. The construction method according to claim 1, wherein no sequencing adapter is added to the filler DNA in step 2.

5. The construction method according to claim 1, wherein the filler DNA in step 2 comprises six polymerase chain reaction (PCR) amplicons of different sizes and CpG densities, five fragments of different CpG densities are methylated, one fragment is unmethylated, and methylated and unmethylated fragments have a mass ratio of 1:1.

6. The construction method according to claim 5, wherein the six PCR amplicons of different sizes and CpG densities are 1 CpG, 5 CpG, 1 CpG, 15 CpG, 20 LCpG, and 20 SCpG.

7. The construction method according to claim 5, wherein the methylated fragments are 1 CpG, 5 CpG, 1 CpG, 15 CpG, and 20 LCpG fragments.

8. The construction method according to claim 5, wherein the unmethylated fragment is a 20 SCpG fragment.

9. The construction method according to claim 1, wherein the filler DNA in step 2 is obtained by performing a PCR with XDNA as a template, purifying and recovering, and methylating, purifying and recovering a resulting PCR fragment.

10. The construction method according to claim 1, wherein the filler DNA in step 2 comprises 50% wt/wt methylated fragments and a 50% wt/wt unmethylated fragment.

11. The construction method according to claim 1, wherein the capturing in step 3 is completed by means of a Diagenode MagMeDIP Kit and a Diagenode iPure Kit V2.

12. The construction method according to claim 1, wherein the amplification and enrichment in step 4 is performed by ligation-mediated polymerase chain reaction (LM-PCR).

13. The construction method according to claim 1, wherein the Illumina sequencing platform in step 5 is one selected from the group consisting of Illumina NextSeq 500, Illumina Hiseq2000, Illumina Hiseq2500, and Illumina Miseq.

14. A method for detecting cfDNA methylation, comprising steps of: sequencing a sequencing library obtained by the construction method according to claim 1 on an Illumina sequencing platform, and bioinformatically analyzing acquired experimental data to know about the cfDNA methylation.

15. A kit for the method for detecting cfDNA methylation according to claim 14, wherein the kit comprises the following components: components for next-generation sequencing (NGS) library preparation, a filler DNA fragment, components for co-immunoprecipitation, methylation capture, and purification and recovery, and components for library enrichment.

16. The kit according to claim 15, wherein the components for NGS library preparation mainly comprise enzymes desired for end repair, A-tailing and adapter ligation in the NGS library preparation and Illumina sequencing platform-specific adapters.

17. The kit according to claim 15, wherein the filler DNA fragment comprises six PCR amplicons of different sizes and CpG densities, five fragments of different CpG densities are methylated, and one fragment is unmethylated;
the six PCR amplicons of different sizes and CpG densities are 1 CpG, 5 CpG, 1 CpG, 15 CpG, 20 LCpG, and 20 SCpG;
the five fragments of different CpG densities are 1 CpG, 5 CpG, 1 CpG, 15 CpG, and 20 LCpG fragments; and
the one fragment is a 20 SCpG fragment.

18. The kit according to claim 17, wherein primers desired for the PCR have nucleotide sequences shown in SEQ ID NOs: 1 to 12.

19. The kit according to claim 15, wherein the components for co-immunoprecipitation comprise buffer reagents desired for the co-immunoprecipitation, an antibody protein, magnetic beads for the methylation capture, and reagents and an Elution Buffer desired for the purification and recovery.

20. The kit according to claim 15, wherein the components for library enrichment comprise an enzyme and a buffer desired for library amplification, and magnetic beads desired for product recovery and purification and fragment screening.
